(19) United States
(12) Patent Application Publication
(10) Pub. No.: US 2025/0025084 A1
(43) Pub. Date: Jan. 23, 2025

(54) SYSTEM AND METHOD OF DELIVERING INTERACTIVE, PERSONALIZED COGNITIVE BEHAVIORAL INTERVENTIONS

(71) Applicants: Vadim Snitkovsky, Rockville, MD (US); Otis Benjamin Young, III, Washington, DC (US); Rosalyn Pham Huynh Mahashin, Kailua, HI (US); Melanie Sage, Buffalo, NY (US)

(72) Inventors: Vadim Snitkovsky, Rockville, MD (US); Otis Benjamin Young, III, Washington, DC (US); Rosalyn Pham Huynh Mahashin, Kailua, HI (US); Melanie Sage, Buffalo, NY (US)

(21) Appl. No.: 18/777,039

(22) Filed: Jul. 18, 2024

Related U.S. Application Data
(60) Provisional application No. 63/514,249, filed on Jul. 18, 2023.

(51) Int. Cl. A61B 5/16; G16H 10/20; G16H 10/60; G16H 50/20

(52) U.S. Cl. CPC A61B 5/165; A61B 5/167; G16H 10/20; G16H 10/60; G16H 50/20

(57) ABSTRACT

A system for automatically producing a personalized cognitive exercise (PCE) includes a user interface; at least one processor; an interactive display; and at least one data storage unit. The user interface can collect data regarding a user's mental state. The processor can execute computer instructions that: perform a pre-assessment, extract, prepare, and format the data to produce formatted data, generate with generative artificial intelligence the PCE from the formatted data and a set of generic cognitive exercises; perform a post-assessment, and assess an impact of the PCE by comparing the post-assessment to the pre-assessment. The interactive display can interactively present the PCE, producing at least one medium selected from the group consisting of text, audio, video, image, and virtual reality (VR). The data storage unit can retrievably store the data and the generic cognitive exercises

### CROSS-REFERENCE TO RELATED APPLICATION

This application claims the benefit of priority of U.S. provisional application No. 63/514,249, filed Jul. 18, 2023, the contents of which are herein incorporated by reference.

### BACKGROUND OF THE INVENTION

The present invention relates to cognitive behavioral therapy for stress and anxiety disorders and, more particularly, to a system and method of delivering interactive, personalized cognitive behavioral interventions.

According to the Anxiety and Depression Association of America (ADAA), anxiety disorders are the most common mental illness in the United States (US), affecting over 40 million adults each year. Furthermore, the National Institute of Mental Health (NIMH) estimated that, in 2021, approximately 57.8 million adults aged 18 or older in the United States struggled with their mental health, representing 22.8% of all U.S. adults. Mediums such as TV, video games, and social media are often used to escape from perceived sources of anxiety but frequently leave the users more anxious/stressed when exiting those mediums.

Personalized cognitive behavioral interventions have shown great promise in improving mental health outcomes. By tailoring therapeutic exercises to an individual's quantifiable anxiety levels and anxiety intensity, as well as their intervention preferences, personalized interventions can increase engagement, improve retention of coping strategies, and potentially lead to faster and more sustained improvements in mental health. However, the challenge lies in creating truly personalized content at scale, which is where artificial intelligence (AI)-driven solutions can play a crucial role.

There are currently no available products on the market that provide completely personalized cognitive exercise content anchored to and referencing the user's life situations. Currently, available smartphone apps in the cognitive behavioral therapy field predominantly rely on providing generic, evidenced-based exercises rather than personalized ones. While a growing number incorporate artificial intelligence (AI) to curate and recommend these exercises, the approach is largely analogous to services like Netflix® and Spotify®, where content is pre-made and filtered down based on user preferences, similar user matching, and historical patterns. These methods result in recommendations that, despite being AI-enhanced, still lack the deep personalization required to truly resonate with an individual's life situations and emotional states. Consequently, these systems often deliver a therapeutic experience that feels impersonal and disconnected, undermining user engagement and the overall effectiveness of the intervention. These systems often fail to achieve their therapeutic potential because they lack a crucial element of personalization, which is essential when addressing mental health. When individuals are distressed, they need to feel understood on a deeply personal level. The use of generic recommendations, even when curated by AI, can feel akin to speaking with a therapist who never acknowledges the specifics of one's situation or relates their advice directly to that individual's unique challenges. This lack of personal connection and relevance can not only render the exercises ineffective but can also exacerbate the user's sense of isolation or misunderstanding, ultimately backfiring on the intended therapeutic goals.

As can be seen, a software system that provides personalized cognitive exercise content to directly reference and cater to an individual's unique life situations is needed.

### SUMMARY OF THE INVENTION

In one aspect of the present invention, a system for automatically producing a personalized cognitive behavioral exercise (PCE) comprises a user interface operative to collect data regarding a user's mental state; at least one processor operative to execute computer instructions that: perform a pre-assessment, extract, prepare, and format the data to produce formatted data, generate with generative artificial intelligence the PCE from the formatted data and a set of generic cognitive exercises; perform a post-assessment, and assess an impact of the PCE by comparing the post-assessment to the pre-assessment; an interactive display operative to interactively present the PCE, wherein the interactive display produces at least one medium selected from the group consisting of text, audio, video, image, and virtual reality (VR); and at least one data storage unit operative to retrievably store the data and the generic cognitive exercises. Furthermore, the system prioritizes user privacy by ensuring all data processing and storage occurs locally on the user's device, without transmitting sensitive information externally.

In another aspect of the present invention, a method for automatically producing a personalized cognitive exercise (PCE) comprises providing the system; administering the pre-assessment; prompting the user data regarding the user's mental state; extracting, preparing, and formatting the data to produce the formatted data; saving the formatted data; generating the PCE from the formatted data and the set of generic cognitive exercises; interactively presenting the PCE to the user; performing the post-assessment; and assessing the impact of the PCE by comparing the post-assessment to the pre-assessment.

The present subject matter is a mental health application for mobile devices that achieves a level of personalization in cognitive behavioral therapy (CBT) exercises that is currently unmatched in the market. The method comprises ingesting data regarding the user's current worries and/or life challenges, as well as context from a user's life using natural language processing methods, and creation of entirely personalized and contextually relevant cognitive exercises. This method, and the system's ability to engage in ongoing, adaptive dialogue, ensures a more effective and engaging therapeutic experience. The context may be obtained either by direct input or passively. The app delivers a multimodal therapeutic experience through various interfaces. The application generates highly personalized cognitive exercises, custom-tailored to the user's needs, and curates exercises selected from an extensive database. These personalized cognitive exercises have been shown to improve mood, help manage anxiety, and reduce stress. The system and method of the present subject matter specifically aim to solve the problem of insufficient personalized cognitive exercise content that can directly reference and cater to an individual's quantifiable anxiety levels. By addressing the critical need for personalization and relevance, our system markedly surpasses the capabilities of existing solutions, providing a therapeutic experience that is both engaging and effective.

These and other features, aspects and advantages of the present invention will become better understood with reference to the following drawings, description, and claims.

### BRIEF DESCRIPTION OF THE DRAWINGS

FIG. 1 is a flow diagram showing a method of user interaction with a behavioral feedback system according to an embodiment of the present invention;

FIG. 2 is a schematic view of the behavioral feedback system of FIG. 1;

FIG. 3 is diagram of the system of FIG. 1, showing relationships between the software and the generative AI and large language model (LLM);

FIG. 4A is a flowchart of a method according to an embodiment of the present invention, illustrating an interaction between a user U and the system S of FIGS. 1-3;

FIG. 4B is a continuation of the flowchart of FIG. 4A;

FIG. 4C is a continuation of the flowchart of FIG. 4A; and

FIG. 5 is another system diagram according to an embodiment of the present invention.

Numbering restarts on each Figure. Therefore, like numbers do not necessarily indicate like components or steps.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, the term “large language model” (LLM) refers to a specific category of language models that are characterized by their extensive size and complexity. For example, a Large Language Model based on a transformer architecture, such as OpenAI's GPT models, Nvidia's Megatron-LM, or Microsoft's Turing-NLG, utilizes massive data sets and scaling of the transformer architecture. For example, the GPT-3 training data set includes results from a massive web crawl. This volume of data allows the expansion of GPT-3 to 175 billion parameters using 96 attention layers, each with a 96×128 dimension head, enabling few or zero-shot training paradigms. By prompting the model with a few response paradigms, the GPT-3 model understands the context, produces results, and can structure its response automatically, without retraining parameters.

Broadly, an embodiment of the present invention provides a system that utilizes artificial intelligence (AI) large language models (LLM) to construct personalized cognitive exercises specific to the user's situation. The personalization increases overall engagement by the user.

For example, if a user expresses anxiety about an upcoming job interview, the LLM might generate a personalized affirmation exercise that incorporates specific details about the user's qualifications and past successes. It could also create a guided visualization exercise that walks the user through a successful interview scenario, drawing on the user's input about the specific company and role they're interviewing for.

The system includes a User Device, i.e., a computing device such as a smartphone, tablet, or computer as the primary interface. In some embodiments, the user device may be provided as multiple independent devices that operate together to prompt and ingest user data and to deliver personalized cognitive exercises. The user device has hardware and software to receive user inputs and display outputs, including audio, video and text. For example, inputs may include free-form audio or text through direct interaction. Direct input may include expressions of the user's current thoughts and feelings. The system also encompasses mechanisms for passive data collection. Passive input may include analysis of digital mediums to which the user grants access, like social media, journal entries, a diary, notes, etc. The user device can output multimodal content, including Cognitive Personalized Exercises via audio, video, and VR. The user device may be equipped with Speech-to-Text for processing spoken words and a processor executing algorithms for analyzing text from various sources. Moreover, the user device generally has local data storage. All activities, including inputs, interactions, and exercise engagements, are stored locally on the user's device. Insights generated, favorites selected, and records of previous exercises are also kept exclusively on the user's device. This approach ensures that personal data remains private, user-controlled, and secure.

The user device also includes hardware and software for presenting the personalized cognitive exercises in the user's preferred format. The exercises may be presented using textual, audio, and visual interfaces. These cognitive exercises are delivered to the user through a multifaceted audio-visual system, allowing for considerable user personalization. The user device may have a Multimodal Interface module. Multimodal user interface modules provide diverse delivery modes for cognitive exercises to suit user preferences and enhance engagement. This multimodal delivery ensures that the exercises are accessible and resonate with the user, fostering a deeper therapeutic experience. An audio module may include text-to-speech capabilities for vocalizing affirmations, coupled with calming, user-selected images. The images may be overlaid with the affirmation text. A video module offers an immersive, user-customizable experience playing serene, user-selected or provided video footage, such as nature scenes. This module may be further enriched with user-configurable, calming, meditative, nature-inspired ambient music, facilitating a highly personalized, harmonious audio-visual experience. This robust, multimodal approach ensures a tranquil experience, promoting mental health and well-being in a uniquely interactive manner.

A virtual reality (VR) module may be integrated into the User Device. The VR module may offer an immersive experience, enabling users to interact with calming environments and visualizations in a more holistic manner. The VR module can present personalized cognitive exercises in an immersive 3D environment. For example, it might create a calming virtual space for meditation exercises, or project affirmations and calming images, all tailored to the user's specific needs and preferences.

The system comprises at least one processor operating a Large Language Model (LLM), a personalization engine, a wellness data collector, a Cognitive Exercise Delivery module, and a feedback optimization engine. In some embodiments, the processor may also operate an ongoing conversational interface. The software's architecture includes algorithms and decision-making processes to ensure that the Exercise Database is effectively utilized; user feedback via the Feedback Optimization Engine is incorporated into future interactions; and the overall system remains responsive and adaptive to the user's evolving psychological needs.

The LLM is generally trained on a comprehensive dataset of cognitive exercises. The LLM is also trained on motivational interviewing techniques, enabling it to engage users in a way that enhances their motivation for change and adherence to the exercises. The LLM may be combined with another generative AI model. This input is fed directly into the LLM, which uses artificial intelligence to parse the input. This input allows the software to assemble completely personalized and highly contextually relevant cognitive exercises the user can perform. The model classifies and maps the input into a series of categories, ranging from categorical anxiety types and well-being rankings to preferences for applicable cognitive and behavioral exercises. Whether through a single interaction or a series of exchanges, the system adapts to provide a highly personalized and effective experience. This interactive approach, mirroring aspects of cognitive behavioral therapy (CBT), enables real-time generation and refinement of personalized cognitive and behavioral exercises. The LLM can analyze and interpret the context and emotions from the user's quantifiable inputs to determine user needs and current emotional state. The generative AI maps user needs to content in an Exercise Database, which contains an array of cognitive behavioral therapy exercises. The LLM may utilize if-then relationships to determine the most appropriate cognitive and behavioral exercises. The system may dynamically generate and curate a list of cognitive and behavioral exercises specifically tailored to the user's unique situation. Users can actively engage with these exercises, benefiting from content that feels bespoke and intimately relevant to their personal experiences. The model can create a curated list of additional cognitive exercises based on relevant situational information discerned from the input. This approach not only constructs personalized exercises that significantly increase user engagement but also creates cognitive anchoring. This makes users feel deeply invested in the process, as the exercises are specifically injected with their particular situations using AI and natural language processing to increase meaning. These personalized exercises resonate deeply with the user, enhancing their efficacy in quantifiably improving mood, managing anxiety, and reducing stress.

The system includes an AI anxiety detection module that analyzes user input to determine specific anxiety subtypes (such as social anxiety, generalized anxiety, or panic disorder) and their severity. This information is used for selecting and tailoring the most appropriate interventions.

An Exercise Database (also known as Corpus), housed in a memory storage device of the system, is a comprehensive library of a wide range of cognitive exercises. It is a foundational resource for generating personalized interventions, offering a rich spectrum of cognitive exercises for enhanced therapeutic engagement. The exercises may include but are not limited to: tailored affirmations, action-driven behaviors, grounding techniques, coping strategies, and methods for shifting one's thoughts. This database may be directly accessed by the user for self-guided therapy and is utilized by the LLM to generate personalized interventions. The interventions align with the user's specific needs and preferences. The corpus may be periodically updated with new exercises and the LLM may be retrained. The user may also engage in a secondary or tertiary recommended exercise.

A decision tree module within the system uses the output from the AI anxiety detection module to navigate a predefined structure of intervention strategies that work best with the specific anxiety categories. This module selects the most appropriate evidence-informed intervention templates from the Exercise Database based on the identified anxiety subtype and severity, ensuring a targeted approach to each user's specific needs.

The system may include an ongoing conversational interface that simulates therapeutic dialogue. The conversational Interface is a software component with subroutines to handle different types of user interactions. For example, subroutines may process spoken or written input via technologies like Speech-to-Text (STT) and Transcription. Within Multimodal Interfaces, subroutines may use Text-to-Speech (TTS) and assemble TTS with Background Music and user pre-selected Background Images. The Conversational Interface enables dynamic, back-and-forth dialogue between the user and the system. The LLM may then further dynamically refine the personalized content based on immediate, real-time user feedback.

A Personalization Engine uses AI algorithms to analyze user input data and to tailor cognitive exercises to the user's specific situations. The Personalization Engine considers user data collected over time, including mood changes and engagement levels. The personalization engine utilizes content from the Exercise Database to ensure relevance and effectiveness of the exercises.

A Wellness Data Collector gathers and analyzes additional user data pre- and post-engagement with the exercises. For example, user input, mood, stress levels, and biometric data may be monitored. This mechanism enhances the Personalization Engine by providing feedback on the user's emotional and physiological responses to the exercises. Logic gates, etc. enable the Wellness Data Collector to interpret data to determine when and how to adjust the output.

Users may be encouraged to provide feedback on the exercises through the app. User reactions and feedback on the exercises are captured by a Feedback Optimization Engine. The feedback contributes to continuous improvement of the personalization process and adaptation of content generated by the LLM. The captured feedback may include exercise ratings. Feedback and data from each interaction cycle enables the system to learn and adapt. This information is used to continuously improve the personalization of future exercises. This ensures that each user's experience is continually refined, making the therapy more effective and personalized over time.

Personalized cognitive exercises are delivered to the user through the Cognitive Exercise Delivery module. Visually presenting personalized cognitive exercises from the LLM through Cognitive Exercise Delivery ensures an engaging and accessible experience.

This app generally prioritizes user privacy. No user content is stored in the cloud, underscoring a commitment to data protection. The app generally does not collect, sell, or share any of the user's personal data. The app functions without transmitting sensitive information externally, even for the purposes of AI training.

The system employs advanced encryption protocols to secure all locally stored data. Users have complete control over their data, including the ability to delete it at any time. The AI models are designed to learn and improve without needing to access or store individual user data, ensuring that the system can evolve and enhance its capabilities while maintaining strict user privacy.

The system employs a privacy-preserving computation module that uses secure enclave technology to isolate sensitive computations. This module also implements differential privacy techniques, adding calibrated noise to any aggregate data to prevent individual user identification while still allowing for system improvements.

In some embodiments, real time input is obtained from the user and/or biofeedback is performed while the user is doing a cognitive exercise, so that the software dynamically adjusts the exercise in progress.

In some embodiments, the present invention includes exercise blending, wherein hybridized exercises are generated and tested to improve user mood and stress levels. Exercise blending involves the AI-driven combination of different cognitive behavioral techniques to create novel, hybridized exercises. For instance, the system might blend elements of mindfulness meditation with cognitive restructuring, creating a unique exercise that guides the user through mindful awareness of anxious thoughts, followed by a structured process for challenging and reframing those thoughts. These blended exercises are then evaluated based on user feedback or biometric data to continually refine and improve their effectiveness.

In some embodiments, user feedback and exercise ratings may be captured relative to the user's situation and used to train the model, improving output for other users with similar situations/sets of inputs.

In some embodiments, the system may include a safety monitoring module operative to utilize artificial intelligence to analyze user input for indications of potential harm to self, harm to others, or being harmed by others; upon detection of such indications, immediately generate and display an alert dialog to the user; provide within the alert dialog direct access to crisis support services, including a one-click option to initiate a text message conversation with a crisis hotline; automatically populate an initial crisis hotline message with contextually relevant information based on the detected indication of harm; and continue monitoring subsequent user interactions to assess a need for additional interventions or support.

To use the inventive system, the user may download and install the application on a compatible user device. Upon launching the app for the first time, the user is guided through a setup process. This may involve creating an account, providing their name, and potentially answering several questions for personalization. The user may be requested to set preferences for interaction modes (audio, text, or VR). The user may link external sources, granting access to the contents for passive data collection. The user may open the software application and express their current thoughts, feelings, or challenges by speaking into a microphone or typing on a keyboard. This input is digested by software with internal artificial intelligence driving an LLM. The LLM generates and recommends a personalized cognitive exercise to the user. These exercises are tailored to address the user's specific situation, leveraging a comprehensive database of CBT strategies.

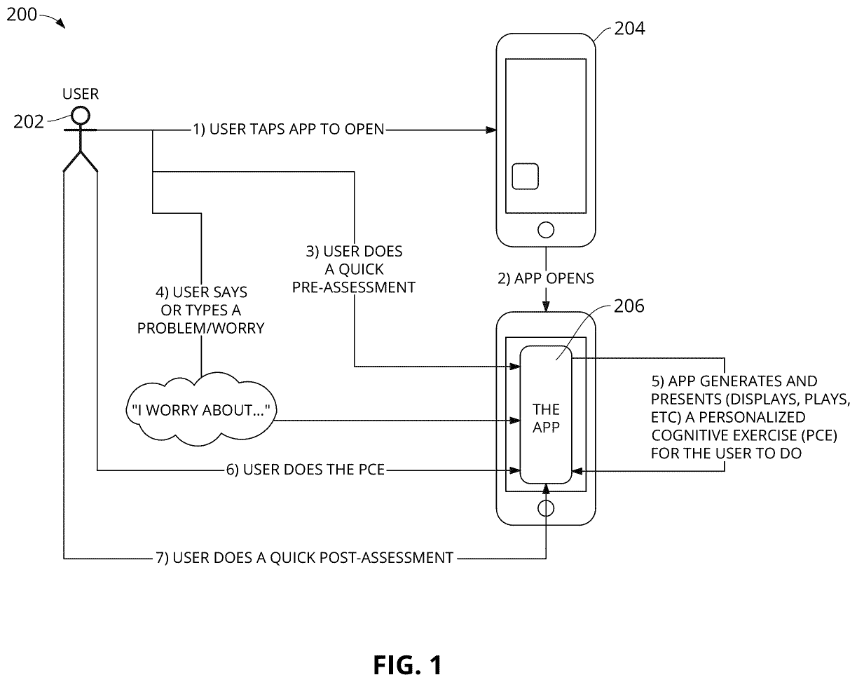

Referring now to FIGS. 1 through 4A and 4B, steps of a method according to an embodiment of the present invention, performed by a user 202, are shown in FIG. 1. The method 200 of utilizing the app 206 comprises the user tapping 1 an icon on the user's device 204 to open the app 2; the user performing a pre-assessment 3; the user expressing thoughts, feelings, or challenges, e.g., a problem or worry; the app generating and presenting a personalized cognitive exercise (PCE) 5; the user performing the PCE 6; and the user performing a post-assessment 7.

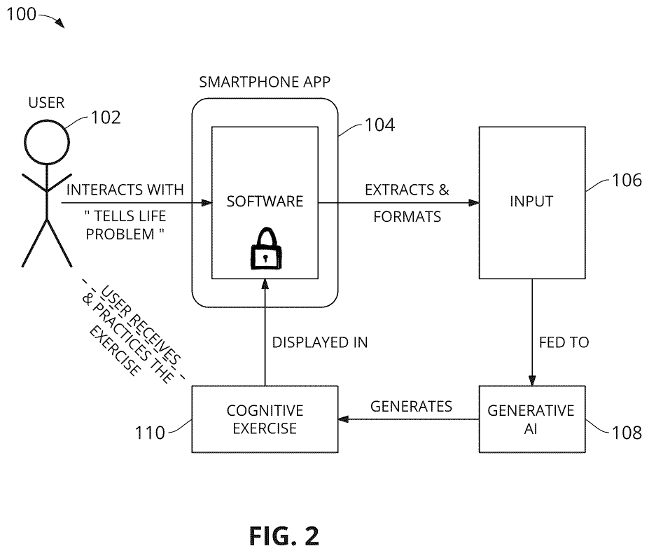

FIG. 2 illustrates a system 100 according to an embodiment of the present invention. The system comprises a user's device 104, an input component 106, a context specific trained LLM 108 hosted at a backend, and a display component, which may be integrated into the device 104. The user 102 interacts with a software application saved to a computer-readable medium, e.g., installed on the user's device 104. The software extracts and formats the input 106 into text content. The formatted input is fed to the LLM 108. The LLM 108 pulls from a list of cognitive exercise categories to generate a personalized cognitive exercise 110. The personalized cognitive exercise is displayed on the user's device 104 for the user 102 to perform.

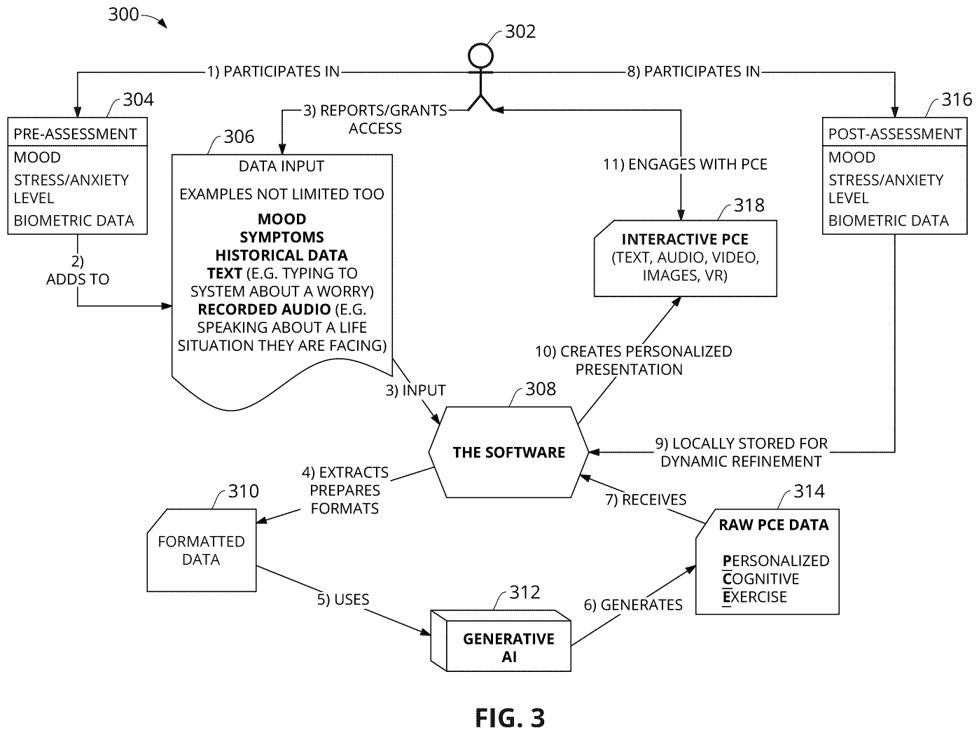

As shown in FIG. 3, the system 300 collects data regarding the participating 1 user's 302 mood, stress or anxiety level, and/or biometric data via a pre-assessment 304 before user engagement 11 with a personalized cognitive exercise. The pre-assessment data is added 2 to data reported or granted access to 3 by the user 302 to produce an input data set 306. The input data set 306 is input 3 into the app software 308, which extracts, prepares, and formats 4 the data to produce formatted data 310. Generative AI 312 uses 5 the formatted data 310 to generate 6 a raw personalized cognitive exercise (PCE) 314. The software 308 receives 7 the PCE and creates a personalized presentation 10 to produce an interactive PCE 183, which may include one or more of text, audio, video, image, and VR. The system 300 collects data regarding mood, stress or anxiety level, and/or biometric data via a post assessment 316 in which the user 302 participates 8 after engagement 11 with the personalized cognitive exercise to assess its impact. The data is locally stored 9 by the software 308 to enable dynamic refinement of the system's performance.

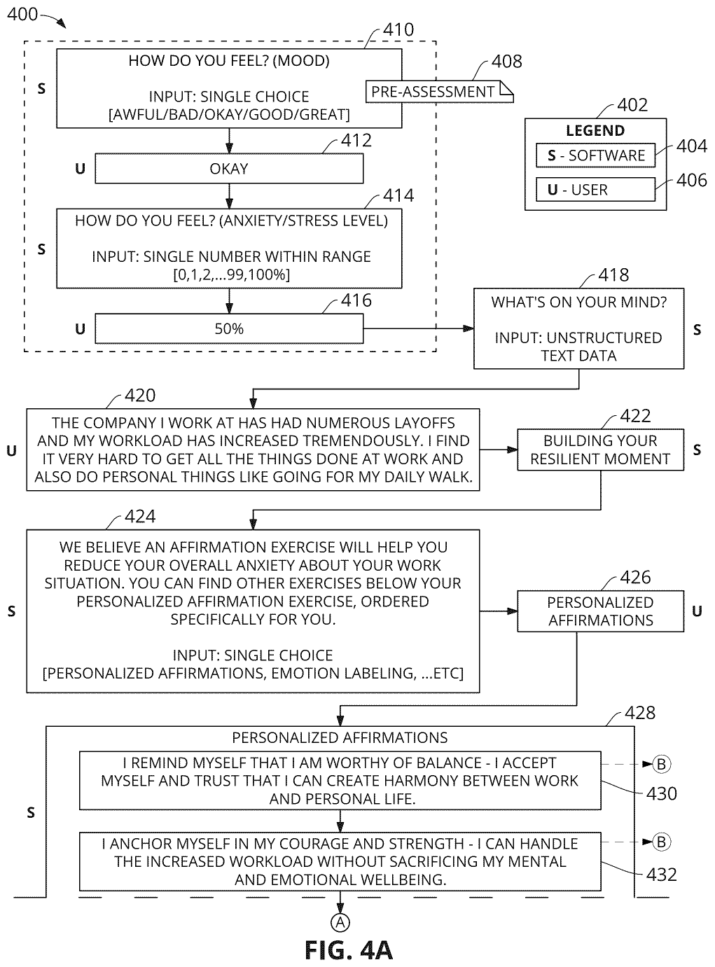

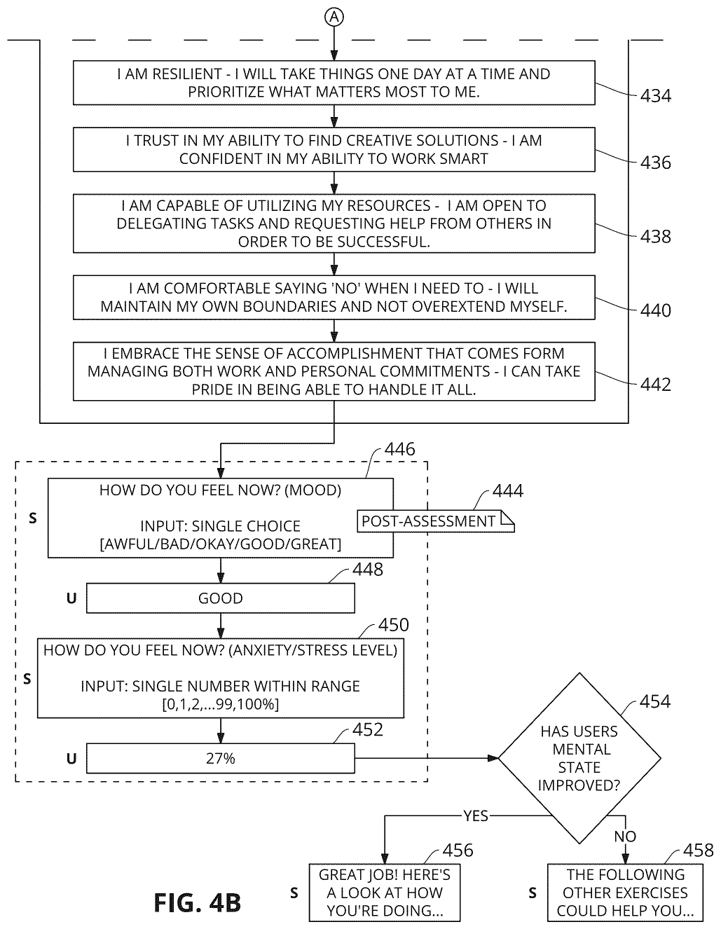

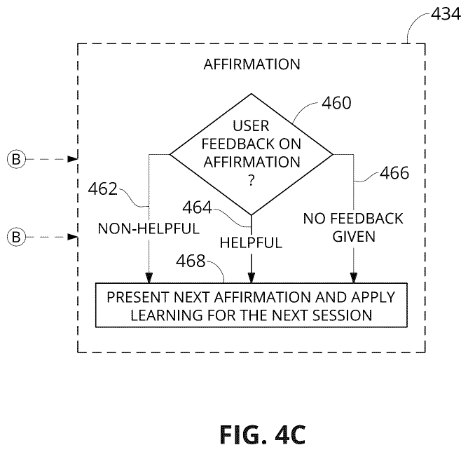

A specific example of an operational method 400 with interactions between the user (U) 406 and the system/software/app (S) 404 is shown in FIGS. 4A, 4B, and 4C. See Legend 402. The app 404 administers a preassessment 408, then prompts 410 the user 406 for input. The app 404 collects the user's 406 response 412 and may prompt 414, 418 the user 406 for additional detail 416, 420. The app 404 processes the data 422 and displays a menu of exercises 424 from which the user 406 selects 426. The app 404 presents the selected exercises 428 which may include, for example, a series of personalized affirmations 430, 432, 434, 436, 438, 440, 442. The app 404 follows with a post-assessment 444, prompting 446, 450 the user 406 for input 448, 452. The app 404 compares 454 the post-assessment 444 results to the pre-assessment 408 results to determine whether the user's mental state has improved. If yes, the app 404 displays encouragement 456. If no, the app 404 displays another menu of exercises 458. As shown in FIG. 4C, a feedback module 460 solicits feedback from the user 406 for at least some of the personalized affirmations 430, 432, 434. The user 406 may report that the affirmation 430, 432 was non-helpful 462 or helpful 464. Alternatively, the user 406 may give no feedback 466. The system then presents a subsequent affirmation and applies the feedback to a learning process (training iteration) 468 for the next session.

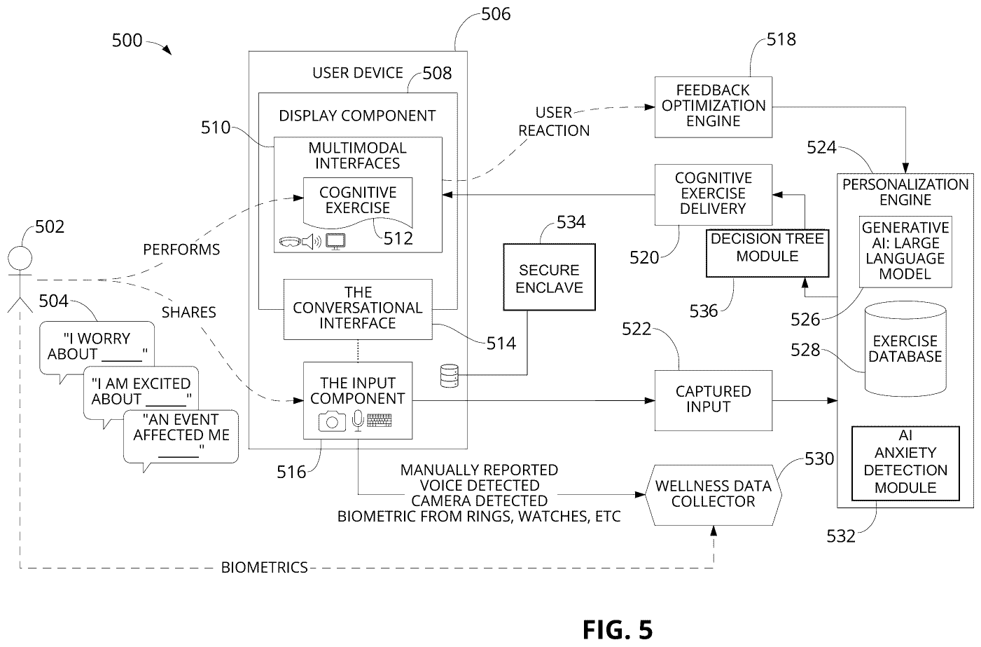

Turning to FIG. 5, a system 500 according to an embodiment of the present invention comprises a user device 506 operated by a user 502 and a backend comprising a feedback optimization engine 518, a personalization engine 524 a captured input module 522, and a wellness data collector 530. The user device comprises a display component 508, and an input component 516, which may include, for example, a camera, a microphone, a keyboard, a ring, and/or a watch. The display component 508 presents cognitive exercises 512 such as affirmations 504 via multimodal interfaces 510, such as a VR apparatus, speaker(s) and video display. A conversational interface 514 interacts with both the display component 508 and the input component 516. The user device 506 may house a secure enclave 534 with a database that stores personal data about the user 502.

The personalization engine 524 comprises a generative AI-driven large language model 526 and an exercise database 528. The feedback optimization engine 518 collects any reaction from the user 502 via the user device 506 and the captured input module 522 collects the user's 502 input from the input component 516 and both feed the personalization engine 524. An AI anxiety detection module 532 within the personalization engine determines the type and level of anxiety exhibited by the user 502. The personalization engine 524 selects cognitive exercises from the exercise database 528 and the AI tailors the exercises, delivering them to display component 508 by way of a decision tree module 536 and the cognitive exercise delivery module 520. The wellness data collector 530 collects biometric data from the user 502 and from the input component 516.

The feedback optimization engine 518 continuously analyzes user reactions and exercise effectiveness, feeding this information back to the personalization engine 524. Simultaneously, the wellness data collector 530 provides user-reported mood data and/or real-time biometric data, allowing the personalization engine 524 to adjust exercises based on the user's self-reported emotional state and/or physiological indicators. The user can select their current mood from a predefined set of options, providing immediate emotional context. This self-reported data can be used in conjunction with or as an alternative to biometric data, depending on the user's preferences and the available inputs. This dual approach allows for a more comprehensive understanding of the user's current state, enabling the personalization engine to tailor exercises more effectively to the user's immediate needs and conditions. This creates a dynamic, responsive system where each component informs the others, resulting in increasingly personalized and effective interventions over time.

Future developments of this system may include integration with other digital health platforms, expansion of the exercise database to include culturally diverse interventions, and the incorporation of advanced natural language understanding to pick up on subtle emotional cues in user input. The system's modular design allows for easy integration of new features and technologies as they become available, ensuring that it can evolve to meet changing user needs and advancements in mental health care.

It should be understood, of course, that the foregoing relates to exemplary embodiments of the invention and that modifications may be made without departing from the spirit and scope of the invention as set forth in the following claims.

What is claimed is:

1. A system for automatically producing a personalized cognitive and/or behavioral exercise (PCE), comprising:
a user interface operative to collect data regarding a user's mental state, including user-reported mood selections and/or biometric data;
at least one processor operative to execute computer instructions that:
perform a pre-assessment,
extract, prepare, and format the data to produce formatted data,
generate with generative artificial intelligence a PCE from the formatted data and a set of generic cognitive and behavioral exercises;
perform a post-assessment, and
assess an impact of the PCE by comparing the post-assessment to the pre-assessment;
an interactive display operative to interactively present the PCE, wherein the interactive display produces at least one medium selected from the group consisting of text, audio, video, image, and virtual reality; and
at least one data storage unit operative to retrievably store the data and the generic cognitive and behavioral exercises.

2. The system of claim 1, wherein the at least one processor is operative to dynamically refine generation of the PCE.

3. The system of claim 1, wherein the user interface is operative to receive the data directly from a user as text or voice-to-text.

4. The system of claim 1, wherein the user interface is operative to receive authorization to access the data from a secondary source.

5. The system of claim 1, further comprising an artificial intelligence (AI) anxiety detection module operative to:
analyze collected user data using machine learning algorithms,
apply a set of criteria for multiple diagnostic anxiety subtypes, and
determine a user's specific anxiety subtype and severity.

6. The system of claim 1, further comprising a decision tree module operative to:
receive an anxiety subtype and severity assessment,
navigate a predefined decision tree structure based on an assessed anxiety subtype and severity, and
select an evidence-informed intervention template from a database of interventions.

7. The system of claim 1, further comprising:
an AI personalization module operative to:
analyze specific statements and contextual information inputted by a user,
utilize natural language processing to extract themes, emotions, and personal details from user input, and
dynamically modify a selected evidence-informed intervention template to create a highly personalized intervention.

8. The system of claim 1, wherein the user interface, the at least one processor, and the interactive display are contained in or coupled to a user's device, and the system further comprises a privacy-preserving computation module operative to:
execute data processing, AI anxiety detection, and intervention selection processes locally on the user's device,
facilitate secure communication with an external large language model (LLM) over an internet connection while ensuring user data and results from the LLM communication are stored and processed locally on the user's device,
ensure that computations involving the user data occur within a secure enclave on the user's device, and
implement differential privacy techniques to protect user privacy in any aggregate statistics and/or logs generated by the system.

9. The system of claim 1, further comprising a personalization engine operative to:
utilize a large language model (LLM) trained on diverse datasets encompassing cognitive behavioral therapy (CBT) exercises, motivational interviewing techniques, mental health scenarios, and user interaction patterns,
generate contextually relevant and highly personalized cognitive and/or behavioral exercises by analyzing user input data,
incorporate situational context and emotional state derived from user data to tailor the cognitive and/or behavioral exercises, and
update and refine the LLM periodically by incorporating new datasets and user feedback.

10. The system of claim 1, further comprising an ongoing conversational interface operative to:
simulate therapeutic dialogue with a user through advanced natural language processing and generation,
process spoken and/or written input via speech-to-text (STT) and text-to-speech (TTS) technologies,
utilize multimodal interfaces to present conversational responses,
dynamically refine the PCE based on immediate real-time user feedback, and
capture and analyze user emotions through the user-reported mood selections and/or sophisticated voice tone analysis and contextual understanding of user input.

11. A method for automatically producing a personalized cognitive and/or behavioral exercise (PCE), comprising:
providing the system of claim 1;
administering the pre-assessment;
prompting a user to submit the data regarding the user's mental state;
extracting, preparing, and formatting the data to produce the formatted data;
saving the formatted data;
generating the PCE from the formatted data and the set of generic cognitive exercises;
interactively presenting the PCE to the user;
performing the post-assessment; and
assessing the impact of the PCE by comparing the post-assessment to the pre-assessment.

12. The method of claim 11, further comprising prompting the user for additional detail regarding the user's mental state, wherein the additional detail is quantifiable.

13. The method of claim 11, further comprising generating a menu of PCEs from which the user selects the PCE to be interactively presented.

14. The method of claim 11, wherein the PCE is selected from the group consisting of tailored affirmations, action-driven behaviors, grounding techniques, coping strategies, methods for shifting one's thoughts, and any combination thereof.

15. The method of claim 11, wherein when the assessing indicates the user's mental state has quantifiably improved between the pre-assessment and the post-assessment, the method further comprises displaying a message of encouragement.

16. The method of claim 11, wherein when the assessing indicates the user's mental state has not quantifiably improved between the pre-assessment and the post-assessment, the method further comprises displaying a menu of other PCEs.
